(19) United States
(12) Patent Application Publication
(10) Pub. No.: US 2023/0191021 A1
(43) Pub. Date: Jun. 22, 2023

(54) INTRAVENOUS SET HAND PUMP

(71) Applicant: CareFusion 303, Inc., San Diego, CA (US)

(72) Inventors: Kelly Kloster HON, Del Mar, CA (US); Suhas HANUMANTHARAO, Karntataka (IN)

(21) Appl. No.: 18/072,183

(22) Filed: Nov. 30, 2022

Related U.S. Application Data
(60) Provisional application No. 63/292,123, filed on Dec. 21, 2021.

(51) Int. Cl. A61M 5/142

(52) U.S. Cl. CPC A61M 5/1424; A61M 2205/586

(57) ABSTRACT

An IV set hand pump includes a body having an egg-shape, a grip portion extending radially outward from the body, an inlet port disposed at a first end of the body and an outlet port disposed at a second end of the body. The IV set hand pump is configured, when squeezed, to increase a fluid flow rate of an IV set downstream of the IV set hand pump beyond a maximum gravity based fluid flow rate of the IV set. IV sets with IV set hand pumps and methods of operating IV set hand pumps are also provided.

### CROSS-REFERENCE TO RELATED APPLICATIONS

This application claims the benefit of priority under 35 U.S.C. § 119 to U.S. Provisional Patent Application Ser. No. 63/292,123, entitled “INTRAVENOUS SET HAND PUMP,” filed on Dec. 21, 2021, the entirety of which is incorporated herein by reference.

### TECHNICAL FIELD

The present disclosure generally relates to intravenous (IV) sets, in particular to hand pumps for IV sets.

### BACKGROUND

Medical treatments often include the infusion of a medical fluid (e.g., blood, plasma, saline) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates as shorter times to blood transfusions have been associated with decreased death risk in trauma patients. Typical IV sets use a cylindrical hand pump that is squeezed by hand to rapidly increase fluid flow rate, resulting in muscle fatigue.

For these reasons, it is desirable to provide an IV set hand pump that is ergonomically shaped with finger grips to reduce hand muscle fatigue and to increase the volume of fluid pushed out by each squeeze of the hand pump.

### SUMMARY

In some embodiments of the disclosure, an IV set hand pump comprises: a body having an egg-shape; a grip portion extending radially outward from the body; an inlet port disposed at a first end of the body; and an outlet port disposed at a second end of the body, wherein the IV set hand pump is configured, when squeezed, to increase a fluid flow rate of an IV set downstream of the IV set hand pump beyond a maximum gravity based fluid flow rate of the IV set.

In some embodiments of the disclosure, an IV set comprises: a first IV tube configured to be coupled to a fluid source; a second IV tube; and an IV set hand pump, comprising: a body having an egg-shape; a grip portion extending radially outward from the body; an inlet port disposed at a first end of the body, the inlet port coupled to the first IV tube; and an outlet port disposed at a second end of the body, the outlet port coupled to the second IV tube, wherein the IV set hand pump is configured, when squeezed, to increase a fluid flow rate of the IV set downstream of the IV set hand pump beyond a maximum gravity based fluid flow rate of the IV set.

In some embodiments of the disclosure, a method of operating an IV set hand pump comprises: coupling a first IV tube of an IV set to a fluid source; coupling a second IV tube of the IV set to a fluid recipient; grasping the IV set hand pump coupled between the first IV tube and the second IV tube, the IV set hand pump comprising: a body having an egg-shape; a grip portion extending radially outward from the body; an inlet port disposed at a first end of the body, the inlet port coupled to the first IV tube; and an outlet port disposed at a second end of the body, the outlet port coupled to the second IV tube; compressing the body to force fluid to flow through the second IV tube at a flow rate greater than one of a maximum gravity based fluid flow rate of the IV set and a maximum infusion flow rate of an infusion pump; expanding the body to pull more fluid from the fluid source into the body; and repeating the compressing and expanding steps to deliver a desired volume of fluid to the fluid recipient.

The foregoing and other features, aspects and advantages of the disclosed embodiments will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a front view of an IV set hand pump, according to aspects of the disclosure.

FIG. 4 depicts a front view of an IV set hand pump held in a hand, according to aspects of the disclosure.

FIG. 5 depicts a perspective view of an IV set with the hand pump of FIG. 3, according to aspects of the disclosure.

FIG. 6 depicts a perspective view of the IV hand pump of FIG. 3 connecting an infusion pump to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

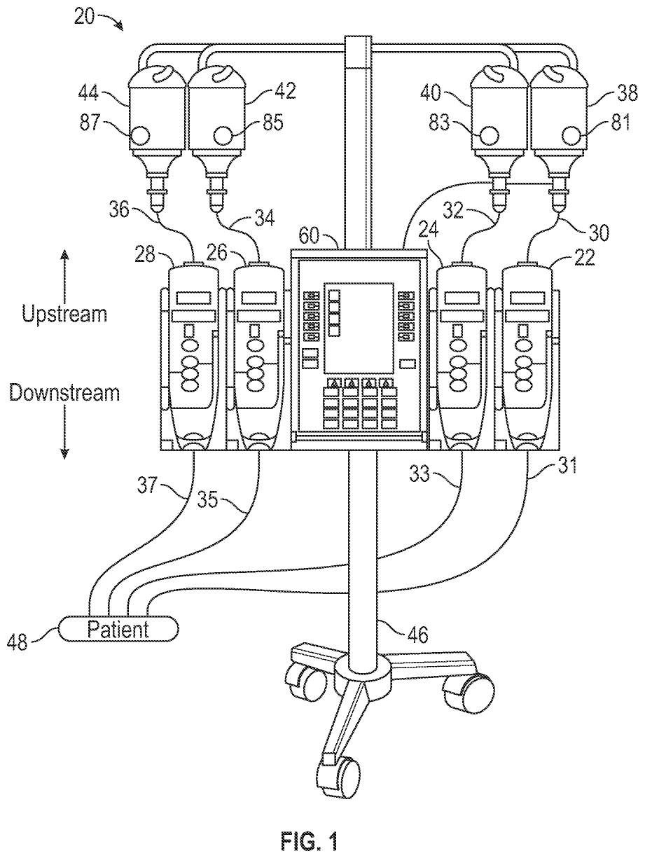

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

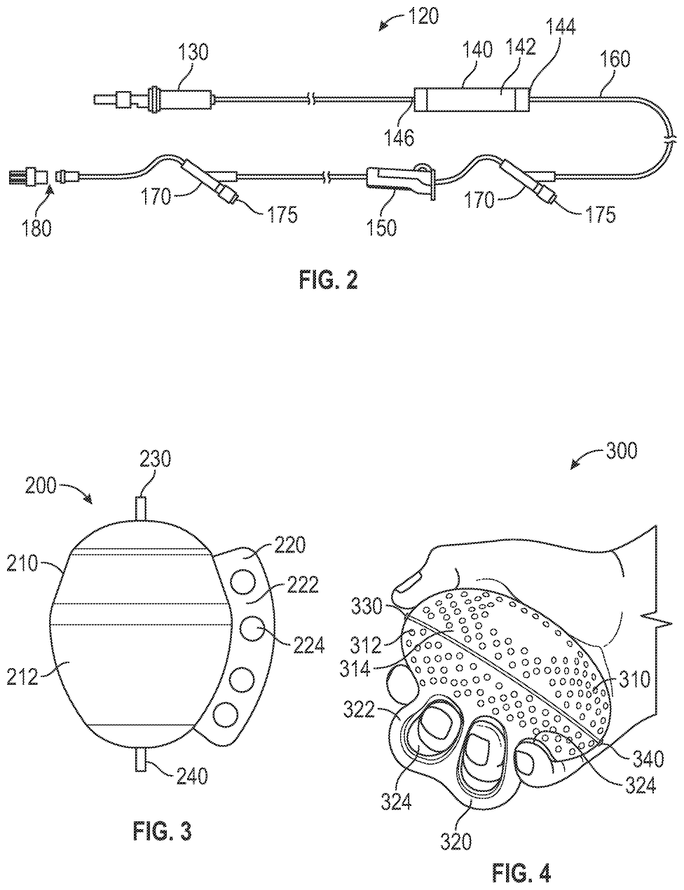

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV fluid bag (e.g., blood bag) via the drip chamber 130 and the luer lock connector 180 is connected to a catheter that is placed into a vein of a patient. Thus, fluid flows from the fluid bag through the drip chamber 130 to the hand pump 140 and through the remainder of the IV set 120 and out of the luer lock connector 180. As the hand pump 140 is squeezed, the volume of fluid contained within a body 142 of the hand pump 140 is forced out the outlet port 144 and downstream through the IV set 120. When the hand pump 140 is released (e.g., stop squeezing), the body 142 of the hand pump 140 reinflates and draws in a new volume of fluid through the inlet port 146. This squeezing cycle can be repeated as often as necessary to move fluid from one or more fluid bags into the patient as quickly as possible.

The hand pump 140 is a typical flexible cylinder that is squeezed by a user continuously with one hand and released before activating or squeezing it again. However, the smooth cylindrical shape of the hand pump 140 provides a non-ergonomic shape to be gripped by a hand and no features to hold fingers in place. Also, the cylinder shape provides squared off internal surfaces that hinder the full amount of fluid within the cylinder from being pushed out with one squeeze.

In aspects of the disclosure, a cylindrical hand pump may be replaced by an egg shaped hand pump with finger grips. The egg-shaped geometry provides that the hand pump fits more naturally in the palm of the user's hand while also providing features through which fingers fit to aid with retraction of the pump once squeezed. This decreases user hand fatigue from pumping fluid and speeds up the retraction cycle in which new fluid is pulled into the expanding body of the hand pump. Also, unlike the cylindrical hand pump 140, the egg-shaped pump has a maximum volume of blood at the bottom of the hand pump and pressuring such an egg shape may release a maximum volume of fluid with one squeeze.

As shown in FIG. 3, an IV set hand pump 200 is provided according to aspects of the disclosure. IV set hand pump 200 may be in the form of an egg-shaped design including a body 210, a grip portion 220, an inlet port 230 and an outlet port 240.

The body 210 may be formed of any suitable flexible material (e.g., polymer, rubber) that is configured to be in contact with a medical fluid (e.g., saline, blood, plasma). The flexible material of the body 210 may be any single material or combination of materials selected or designed to provide a desired amount of flexibility and life-cycle usage. In aspects of the disclosure, the egg shape of the body 210 provides curved interior surfaces that may decrease or eliminate dead areas that retain fluid during fluid outflow from the body 210. For example, in the cylinder shape of hand pump 140, the orthogonal (e.g., squared off) surfaces between the side wall and the end walls provides dead areas in the corner portions that may retain fluid even when the hand pump 140 is fully squeezed, thus decreasing the volume of fluid that can be forced out quickly. By contrast, the curved interior surfaces of the body 210 provide an easier path for the fluid to flow along, thus allowing a higher volume of fluid to be forced out of the body 210 with each squeeze.

The grip portion 220 may extend radially outward from an outer surface 212 of the body 210. The grip portion 220 may include a shaped surface 222 having one or more grip members 224 sized and shaped to be engaged (e.g., receive fingers). The grip portion 220 may have any suitable number of grip members 224. For example, as shown in FIG. 3, there are four grip members 224 in the form of circular openings disposed within the shaped surface 222 in an arcuate fashion, thus closely aligning with the position of all four fingers of a user's hand when the IV set hand pump 200 is gripped. Each grip member 224 may have any suitable size and shape (e.g., circular opening, oblong opening, arcuate groove). Also, the user's thumb may be in contact with the body 210 when the user's fingers are received by the grip members 224. In aspects of the disclosure, the grip members may be grooves or channels in the outer surface for fingers to slide into and/or grip onto. In aspects of the disclosure, the grip members 224 and/or the body 210 may be configured to be gripped by an automated device, such as a robotic arm or mechanical pump fingers, for example.

As shown in FIG. 4, an IV set hand pump 300 is provided according to aspects of the disclosure. IV set hand pump 300 may be in the form of an egg-shaped design including a body 310, a grip portion 320, an inlet port 330 and an outlet port 340. The body 310 may be formed of any suitable flexible material (e.g., polymer, rubber) that is configured to be in contact with a medical fluid (e.g., saline, blood, plasma). The flexible material of the body 310 may be any single material or combination of materials selected or designed to provide a desired amount of flexibility and life-cycle usage.

The body 310 may include texture members 312 to provide users with an easy grip, a comfortable grip, a stimulating grip and/or a non-slip grip. For example, the texture members 312 shown in FIG. 4 are bumps extending out from an outer surface 314 of the body 310. The texture members 312 may be sized and shaped in any suitable manner (e.g., bumps, raised lines, non-slip coatings).

The grip portion 320 may extend radially outward from the body 310. The grip portion 320 may include a shaped surface 322 having one or more grip members 324 sized and shaped to receive one or more gripping elements (e.g., fingers). The grip portion 320 may have any suitable number of grip members 324. For example, as shown in FIG. 4, there are four grip members 324 in the form of two central oblong openings and two outer arcuate grooves disposed within and/or on the shaped surface 322 in an arcuate fashion about the body 310, thus closely aligning with the position of all four fingers of a user's hand when the IV set hand pump 300 is gripped. Each grip member 324 may have any suitable size and shape (e.g., circular opening, oblong opening, arcuate groove). Also, the user's thumb may be in contact with the body 310 when the user's fingers are inserted into or received by the grip members 324, as shown in FIG. 4. In aspects of the disclosure, the grip members 324 and/or the body 310 may be configured to be gripped by an automated device, such as a robotic arm or mechanical pump fingers, for example.

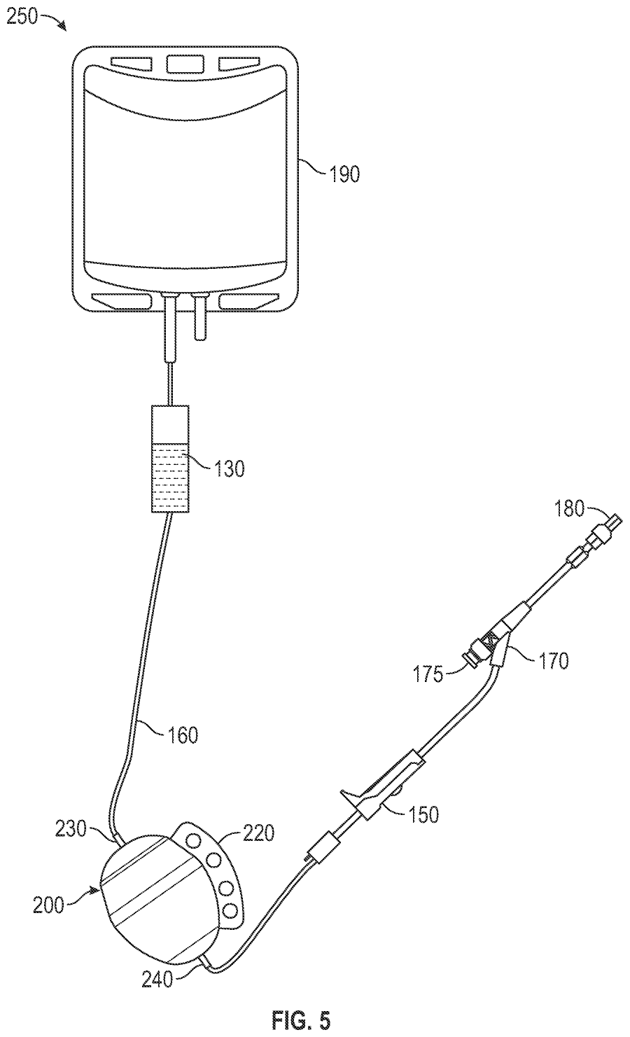

As shown in FIG. 5, the IV set hand pump 200 may be part of an IV set 250. The IV blood set 250 may include a fluid bag 190, a drip chamber 130, an IV set hand pump 200, a roller clamp 150, a Y-junction 170 having a needleless port 175 and a luer connector 180, all coupled together by IV tubing 160. IV set hand pump 300 or any other desired hand pump may be used in place of IV set hand pump 200 in the IV set 250, for example.

In aspects of the disclosure, the IV set hand pump 200, 300 may be a passive in-line device when not activated (e.g., being squeezed) in which fluid from the fluid bag 190 is received via the drip chamber 130 into the inlet port 230, 330 and the fluid passes through the body 210, 310 and out the outlet port 240, 340 at a flow rate controlled by any of gravity, the volume of fluid in the fluid bag 190 and/or the roller clamp 150. In use, when the IV set hand pump 200, 300 is squeezed, the volume of fluid that has accumulated within the body 210, 310 during the passive flow phase may be fully forced out of the outlet port 240, 340 very rapidly while the body 210, 310 collapses/contracts.

Next, the user's hand may be opened where the user's fingers pull outward on one or more grip members 224, 324 to rapidly open/expand the body 210, 310 back to an expanded condition. This rapid expansion of the body 210, 310 may cause a suction effect upstream, thus causing more fluid to flow from the fluid bag 190/drip chamber 130 into the IV set hand pump 200, 300 quickly (e.g., more quickly than gravity allows). Thus, continuous squeezing and opening of the IV set hand pump 200, 300 may cause all of the fluid to be rapidly pulled out of the fluid bag 190 and pushed into a desired receptacle (e.g., a patient's arm).

In aspects of the disclosure, the IV set hand pump 200, 300 may be a disposable component of the IV set 250. For example, the IV set hand pump 200, 300 may be a standalone component that may be added to an existing IV set. As another example, the IV set hand pump 200, 300 may be an integral component of the IV set 250 where the body 210, 310 may be coupled in line via IV tubing 160 to the drip chamber 130 and the roller clamp 150. Accordingly, the disposable IV set hand pump 200, 300 would be used for the life of the IV set only (e.g., 24 hours, 72 hours, 7 days), whereafter the IV set hand pump 200, 300 would be disposed of along with the associated IV set 250. In aspects of the disclosure, the IV set hand pump 200, 300 may be coupled anywhere in line within the IV set 250, such as directly to the fluid bag 190 in place of a drip chamber 130, for example.

In use, the IV set 250 with the disposable IV set hand pump 200, 300 is coupled to a fluid container (e.g., fluid bag 190) containing a medical fluid (e.g., blood). In an unactuated state, IV set hand pump 200, 300 has an uncompressed body 210, 310, thus allowing the fluid to flow through the IV set 250 at a rate set by a flow controller (e.g., roller clamp 150). When quicker fluid flow is needed, the IV set hand pump 200, 300 may be compressed (e.g., squeezed) by hand, thus squeezing the fluid out of the body 210, 310 quickly.

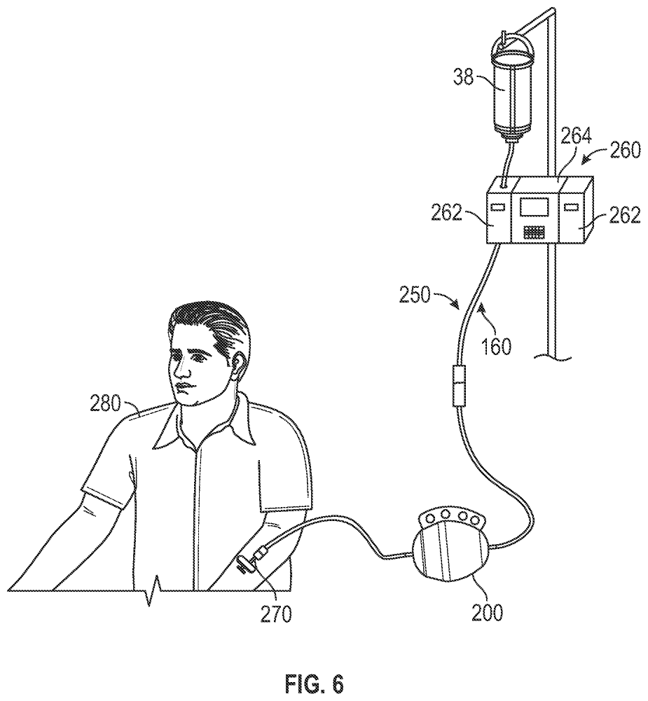

As shown in FIG. 6, the IV set 250 and IV set hand pump 200, 300 may be coupled to a fluid source 38, to an infusion pump 260 system having two infusion pumps 262 and a controller 264, and to a catheter 270 inserted into a patient 280. Here, if the maximum fluid flow rate from the infusion pump system 260 is not sufficient, the IV set hand pump 200, 300 may be activated (e.g., squeezed and released rhythmically) to force the fluid to flow more quickly. For example, the IV set 250 may be quickly uncoupled from the infusion pump system 260 before using the IV set hand pump 200, 300 and/or the infusion pump system 260 may be set to neutral to allow unimpeded fluid flow from the fluid source 38 before using the IV set hand pump 200, 300.

In aspects of the disclosure, the IV set hand pump 200, 300 may include any suitable fastener to couple the IV set hand pump 200, 300 to an IV pole 46, a bed, an operating table and the like. For example, the fastener may be a cradle, a hangar, a hook, Velcro®, adhesive, and/or any other suitable fastener. In aspects of the disclosure, the IV set hand pump 200, 300 may be configured to simply hang in line with an IV set (e.g., IV set 250) via IV tubing (e.g., IV tubing 160). Here, the IV set hand pump 200, 300 may be positioned on the IV set to maximize accessibility and/or to keep the IV set hand pump 200, 300 out of specific work areas (e.g., right above patient).

In aspects of the disclosure, any of the above-described elements or features of IV set hand pump 200 may be used with IV set hand pump 300 and any elements or features of IV set hand pump 300 may be used with IV set hand pump 200.

In aspects of the disclosure, the geometry and/or shape of the body may be varied based on hand size and/or number of finger grips. For example, there may be small, medium and large sizes for use by different sized users. In aspects of the disclosure, the shaped portion and the grip members of the grip portion may be sized and shaped in any suitable manner. In aspects of the disclosure, the flow rates may be configured based on size/shape of the body and diameter of the inlet ports and outlet ports.

In aspects of the disclosure, drugs may be administered to a patient quickly via a manual hand pump that forces the drugs via the IV line at the rate with which user is pumping.

In one or more embodiments, an IV set hand pump comprises: a body having an egg-shape; a grip portion extending radially outward from the body; an inlet port disposed at a first end of the body; and an outlet port disposed at a second end of the body, wherein the IV set hand pump is configured, when squeezed, to increase a fluid flow rate of an IV set downstream of the IV set hand pump beyond a maximum gravity based fluid flow rate of the IV set.

In aspects of the disclosure, an interior of the body comprises curved interior surfaces. In aspects of the disclosure, the curved interior surfaces are configured to one of decrease and eliminate dead areas that retain fluid during fluid outflow from the body through the outlet port. In aspects of the disclosure, the grip portion comprises a shaped surface having one or more grip members. In aspects of the disclosure, the one or more grip members are openings disposed in an arcuate pattern on the shaped surface, each opening sized and shaped to receive a finger. In aspects of the disclosure, the one or more grip members are grooves, each groove sized and shaped to receive a finger. In aspects of the disclosure, the one or more grip members comprise an oblong opening disposed in the shaped surface and an outer arcuate groove disposed on the shaped surface. In aspects of the disclosure, there are two oblong openings configured to receive two interior fingers of a hand and two outer arcuate grooves configured to receive two outer fingers of the hand.

In aspects of the disclosure, the one or more grip members are configured to be gripped by one of a robotic arm and mechanical pump fingers. In aspects of the disclosure, the body is configured to be gripped by one of a robotic arm and mechanical pump fingers. In aspects of the disclosure, the body is formed of a flexible material configured to be in contact with a medical fluid. In aspects of the disclosure, an exterior surface of the body comprises texture members. In aspects of the disclosure, the texture members are bumps. In aspects of the disclosure, the texture members are raised lines. In aspects of the disclosure, the texture members are non-slip coated areas.

In one or more embodiments, an IV set comprises: a first IV tube configured to be coupled to a fluid source; a second IV tube; and an IV set hand pump, comprising: a body having an egg-shape; a grip portion extending radially outward from the body; an inlet port disposed at a first end of the body, the inlet port coupled to the first IV tube; and an outlet port disposed at a second end of the body, the outlet port coupled to the second IV tube, wherein the IV set hand pump is configured, when squeezed, to increase a fluid flow rate of the IV set downstream of the IV set hand pump beyond a maximum gravity based fluid flow rate of the IV set.

In aspects of the disclosure, the grip portion comprises a shaped surface having one or more grip members comprising at least one of a circular opening, an oblong opening and an outer arcuate groove, wherein each grip member is sized and shaped to receive a finger of a hand. In aspects of the disclosure, the body is formed of a flexible material configured to be in contact with a medical fluid, and wherein texture members are disposed on an exterior surface of the body, the texture members comprising one of bumps, raised lines and non-slip coated areas.

In one or more embodiments, a method of operating an IV set hand pump comprises: coupling a first IV tube of an IV set to a fluid source; coupling a second IV tube of the IV set to a fluid recipient; grasping the IV set hand pump coupled between the first IV tube and the second IV tube, the IV set hand pump comprising: a body having an egg-shape; a grip portion extending radially outward from the body; an inlet port disposed at a first end of the body, the inlet port coupled to the first IV tube; and an outlet port disposed at a second end of the body, the outlet port coupled to the second IV tube; compressing the body to force fluid to flow through the second IV tube at a flow rate greater than one of a maximum gravity based fluid flow rate of the IV set and a maximum infusion flow rate of an infusion pump; expanding the body to pull more fluid from the fluid source into the body; and repeating the compressing and expanding steps to deliver a desired volume of fluid to the fluid recipient.

In aspects of the disclosure, the method further comprises replacing a typical cylindrical hand pump with the IV set hand pump, wherein the IV set hand pump reduces user hand fatigue and increases fluid flow rate to reduce fluid transfusion time.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) set hand pump comprising:
a body having an egg-shape;
a grip portion extending radially outward from the body;
an inlet port disposed at a first end of the body; and
an outlet port disposed at a second end of the body,
wherein the IV set hand pump is configured, when squeezed, to increase a fluid flow rate of an IV set downstream of the IV set hand pump beyond a maximum gravity based fluid flow rate of the IV set.

2. The IV set hand pump of claim 1, wherein an interior of the body comprises curved interior surfaces.

3. The IV set hand pump of claim 2, wherein the curved interior surfaces are configured to one of decrease and eliminate dead areas that retain fluid during fluid outflow from the body through the outlet port.

4. The IV set hand pump of claim 1, wherein the grip portion comprises a shaped surface having one or more grip members.

5. The IV set hand pump of claim 4, wherein the one or more grip members are openings disposed in an arcuate pattern on the shaped surface, each opening sized and shaped to receive a finger.

6. The IV set hand pump of claim 4, wherein the one or more grip members are grooves, each groove sized and shaped to receive a finger.

7. The IV set hand pump of claim 4, wherein the one or more grip members comprise an oblong opening disposed in the shaped surface and an outer arcuate groove disposed on the shaped surface.

8. The IV set hand pump of claim 7, wherein there are two oblong openings configured to receive two interior fingers of a hand and two outer arcuate grooves configured to receive two outer fingers of the hand.

9. The IV set hand pump of claim 4, wherein the one or more grip members are configured to be gripped by one of a robotic arm and mechanical pump fingers.

10. The IV set hand pump of claim 1, wherein the body is configured to be gripped by one of a robotic arm and mechanical pump fingers.

11. The IV set hand pump of claim 1, wherein the body is formed of a flexible material configured to be in contact with a medical fluid.

12. The IV set hand pump of claim 1, wherein an exterior surface of the body comprises texture members.

13. The IV set hand pump of claim 12, wherein the texture members are bumps.

14. The IV set hand pump of claim 12, wherein the texture members are raised lines.

15. The IV set hand pump of claim 12, wherein the texture members are non-slip coated areas.

16. An intravenous (IV) set comprising:
a first IV tube configured to be coupled to a fluid source;
a second IV tube; and
an IV set hand pump, comprising:
a body having an egg-shape;
a grip portion extending radially outward from the body;
an inlet port disposed at a first end of the body, the inlet port coupled to the first IV tube; and
an outlet port disposed at a second end of the body, the outlet port coupled to the second IV tube,
wherein the IV set hand pump is configured, when squeezed, to increase a fluid flow rate of the IV set downstream of the IV set hand pump beyond a maximum gravity based fluid flow rate of the IV set.

17. The IV set of claim 16, wherein the grip portion comprises a shaped surface having one or more grip members comprising at least one of a circular opening, an oblong opening and an outer arcuate groove, wherein each grip member is sized and shaped to receive a finger of a hand.

18. The IV set of claim 16, wherein the body is formed of a flexible material configured to be in contact with a medical fluid, and wherein texture members are disposed on an exterior surface of the body, the texture members comprising one of bumps, raised lines and non-slip coated areas.

19. A method of operating an intravenous (IV) set hand pump, the method comprising:
coupling a first IV tube of an IV set to a fluid source;
coupling a second IV tube of the IV set to a fluid recipient;
grasping the IV set hand pump coupled between the first IV tube and the second IV tube, the IV set hand pump comprising:
a body having an egg-shape;
a grip portion extending radially outward from the body;
an inlet port disposed at a first end of the body, the inlet port coupled to the first IV tube; and
an outlet port disposed at a second end of the body, the outlet port coupled to the second IV tube;
compressing the body to force fluid to flow through the second IV tube at a flow rate greater than one of a maximum gravity based fluid flow rate of the IV set and a maximum infusion flow rate of an infusion pump;
expanding the body to pull more fluid from the fluid source into the body; and
repeating the compressing and expanding steps to deliver a desired volume of fluid to the fluid recipient.

20. The method of claim 19, further comprising:
replacing a typical cylindrical hand pump with the IV set hand pump, wherein the IV set hand pump reduces user hand fatigue and increases fluid flow rate to reduce fluid transfusion time.
